(19) United States
(12) Patent Application Publication
(10) Pub. No.: US 2003/0181870 A1
(43) Pub. Date: Sep. 25, 2003

(54) Needle device

(75) Inventors: Peter W. Bressler, Philadelphia, PA (US); John Coleman, Philadelphia, PA (US); Mathieu Turpault, Berwyn, PA (US)

Correspondence Address: THE WEBB LAW FIRM/BECTON, DICKINSON AND COMPANY, 700 KOPPERS BUILDING, 436 SEVENTH AVENUE, PITTSBURGH, PA, 15219, US

(73) Assignee: Becton, Dickinson and Company

(21) Appl. No.: 10/391,992

(22) Filed: Mar. 19, 2003

Related U.S. Application Data
(60) Provisional application No. 60/365,436, filed on Mar. 19, 2002.

(51) Int. Cl. A61M 5/178

(52) U.S. Cl. 604/263

(57) ABSTRACT

The present invention is directed to a shieldable blood collection set as well as a needle assembly for use therein. The needle assembly includes a needle cannula having a puncture tip at a distal end thereof; a hub which supports the needle cannula; and a shield axially movable between a non-shielding position, in which the puncture tip of the needle cannula is exposed from the shield, and a shielding position in which the shield covers the puncture tip. A pair of extendable members interconnects the hub and the shield. The pair of extendable members are moveable between a mating position forming a unitary structure; a lateral position forming a pair of wings extending from opposing sides of the needle assembly; and an extended position causing the shield to move from the non-shielding position to the shielding position.

### CROSS REFERENCE TO RELATED APPLICATIONS

This application claims the benefit of U.S. Provisional Application No. 60/365,436 filed Mar. 19, 2002.

### BACKGROUND OF THE INVENTION

1. Field of the Invention

The present invention relates to blood collection sets for safe and convenient handling of needles. More particularly, the present invention relates to a blood collection set including a needle assembly having a low profile forward moving safety shield for protection from a used needle tip.

2. Description of Related Art

Disposable medical devices having medical needles are used for administering medication or withdrawing fluid from the body of a patient. Such disposable medical devices typically include blood collecting needles, fluid handling needles, and assemblies thereof. Current medical practice requires that fluid containers and needle assemblies used in such devices be inexpensive and readily disposable. Consequently, existing blood collection devices typically employ some form of durable, reusable holder on which detachable and disposable medical needles and fluid collection tubes may be mounted. A blood collection device of this nature may be assembled prior to use and then disassembled after use. Thus, these blood collection devices allow repeated use of a relatively expensive holder upon replacement of relatively inexpensive medical needles and/or fluid collection tubes. In addition to reducing the cost of collecting blood specimens, these blood collection devices help minimize the production of hazardous waste material.

A blood collection device or intravenous (IV) infusion device typically includes a needle cannula having a proximal end, a pointed distal end, and a lumen extending therebetween. The proximal end of the needle cannula is securely mounted in a plastic hub defining a central passage that communicates with the lumen extending through the needle cannula. A thin, flexible thermoplastic tube is connected to the hub and communicates with the lumen of the needle cannula. The end of the plastic tube remote from the needle cannula may include a fixture for connecting the needle cannula to a blood collection tube or other receptacle. The specific construction of the fixture will depend upon the characteristics of the receptacle to which the fixture is to be connected.

In order to reduce the risk of incurring an accidental needle-stick wound, protection of used needle cannulas becomes important. With concern about infection and transmission of diseases, methods and devices to enclose or cover the used needle cannula have become very important and in great demand in the medical field. For example, needle assemblies commonly employ a safety shield that can be moved into shielding engagement with a used needle cannula to minimize risk of an accidental needle stick.

Some needle safety shields are referred to as “tip guards” and include a small rigid guard that may be telescoped along the length of the needle cannula and extended over the pointed distal end of the needle cannula for protection. Such conventional tip guards may include some form of tether for limiting the travel of the tip guard to the length of the needle cannula. An example of the foregoing is disclosed by U.S. Pat. No. 5,176,655 to McCormick et al. The McCormick et al. patent discloses the use of flexible loop-like straps for limiting the distal movement of a tip guard.

Needle shields that incorporate movable tip guards are typically manually actuated. For example, U.S. Pat. Nos. Re 36,447 and Re 36,398, both to Byrne et al., disclose a safety device for a hypodermic needle that includes a plastic sheath, which is used to cover the puncture tip of the needle. The plastic sheath incorporates a thumb guard, which the user of the safety device may grasp to move the plastic sheath to a position covering the puncture tip of the needle cannula. U.S. Pat. No. 5,951,525 to Thorne et al. discloses a manually operated safety needle apparatus that includes two pairs of opposed legs adapted to move the tip guard of the apparatus to a position covering the used needle cannula. U.S. Pat. Nos. 5,562,637 and 5,562,636, both to Utterburg, disclose a rectangular needle protector sheath for use with a needle cannula that may be extended over the needle cannula after its use. Other prior art devices, such as those disclosed by U.S. Pat. Nos. 5,290,264 to Utterberg and 5,192,275 to Burns provide “grippable” members attached to the tip guards to facilitate moving the tip guards to a position covering the puncture tip of a needle cannula. In addition to providing gripping members for moving the tip guards, prior art devices in this area often include flexible wings, which are used as means for securing the needle assemblies to the body of a patient during a medical procedure. Examples of “winged” needle assemblies may found in U.S. Pat. No. 5,120,320 to Fayngold; and U.S. Pat. Nos. 5,154,699; 5,088,982; and 5,085,639 all to Ryan. Other prior art in this area includes U.S. Pat. Nos. 5,266,072 and 5,112,311, both to Utterberg et al., which also disclose guarded winged needle assemblies.

Conventional tip guards, such as those discussed hereinabove, often include a structure that lockingly engages over the pointed distal end of the used needle cannula to prevent a re-exposure of the needle cannula. The structure for preventing the re-exposure of the needle cannula may include a metallic spring clip or a transverse wall formed integrally with one end of the tip guard. An example of a metallic spring clip is disclosed by the McCormick et al. patent discussed previously.

Conventional tip guards, such as those discussed hereinabove, often further require extensive mechanics for positioning the tip guard over the needle cannula. This results in complex arrangements that are costly to manufacture and assemble. Additionally, operation of the needle assemblies to move the tip guard into the proper position over the pointed distal end of the needle cannula requires substantial manual manipulation by the user of the device, exposing the user to potential needle-stick wounds.

In view of the foregoing, a need exists for a blood collection set including a shieldable needle assembly that achieves secure and effective shielding of a used needle cannula which is simple and inexpensive to manufacture and easy to operate.

### SUMMARY OF THE INVENTION

The present invention is directed to a shieldable blood collection set, as well as a needle assembly for use in a blood collection set. The needle assembly includes a needle cannula having a proximal end and a distal end with a puncture tip, and a hub member having a proximal end and a distal end, supporting the proximal end of the needle cannula. The needle assembly further includes a shield member axially movable with respect to the hub member and the needle cannula, between a non-shielding position in which the puncture tip of the needle cannula is exposed from the shield member, and a shielding position in which the shield member covers the puncture tip of the needle cannula. The needle assembly also includes a pair of extendable members interconnecting the hub member and the shield member. The extendable members include structure which is capable of spreading to form a pair of wings extending from opposing sides of the needle assembly, and extending to cause the shield member to move from the retracted position to the extended position. Desirably, the extendable members are also capable of joining to form a unitary structure, such as for insertion of the needle device. In particular, the pair of extendable members are moveable between a mating position in which they form a unitary structure such as a dorsal fin, a lateral position in which they are spread apart to form a pair of wings extending from opposing sides of the needle assembly, and an extended position in which they are extended to cause the shield member to move from the non-shielding position to the shielding position. Desirably, the pair of extendable members are formed of a resilient flexible material.

Each of the pair of extendable members desirably includes a proximal extension and a distal extension, with each of the proximal extensions being connected to opposing lateral sides of the hub member and each of the distal extensions being connected to opposing lateral sides of the shield member. Such connections desirably act as living hinges. Moreover, each pair of extendable members desirably includes an extendable bridge having structure capable of extending to move the shield member from the non-shielding position to the shielding position. The structure of each of the extendable bridges may further include a partial cut-away portion forming a living hinge, which provides for extension of the bridge and movement of the shield member.

In particularly desirable embodiments, the needle assembly includes means for preventing the shield member from moving in an axial direction between the shielding position and the non-shielding position, by locking the pair of extendable members in the extended position. For example, the extendable bridges may include a locking mechanism, and desirably include first and second locking mechanisms, with the first locking mechanisms engagable with each other and the second locking mechanisms engagable with each other. Alternatively, or in addition thereto, the shield member may include a tip guard assembly for locking the shield member in the shielding position. Such a tip guard assembly may include a tip guard housing formed from a plastic material, and a metallic spring clip being mounted to the housing which is biased against the needle cannula when the shield member is in the non-shielding position and which is resiliently moved over the distal end of the needle cannula when the shield member is in the shielding position.

In a further embodiment, the present invention is directed to a shieldable blood collection set which includes a needle assembly as described above, a flexible tube connected to a proximal end of a hub member of the needle assembly, and a fixture connected to the opposing end of the flexible tube for connecting the blood collection set to a receptacle. In particular, the needle assembly includes a needle cannula, a hub supporting the needle cannula, a shield in axial alignment with the hub and movable between a non-shielding position and a shielding position, and a pair of extendable members movable between a mating position forming a unitary structure, a lateral position forming a pair of wings, and a extended position for causing the shield to move from the non-shielding position to the shielding position.

Desirably, the shield is in the non-shielding position and is adjacent the hub when the pair of extendable members are in the mating position and/or the lateral position, and the shield is in the shielding position when the pair of extendable members are extended to the extended position.

### DESCRIPTION OF THE DRAWINGS

FIG. 1 is a perspective view of a needle assembly in accordance with the present invention, shown in a non-shielding position;

FIG. 2 is a perspective view of the needle assembly of FIG. 1, shown in a sampling position;

FIG. 3 is a perspective view of the needle of FIG. 1, shown in an shielding position;

FIG. 4 is a cross-sectional view taken along line IV-IV of FIG. 3;

FIG. 5 is a cross-sectional view taken along line V-V of FIG. 3;

FIG. 6 is a perspective view of a blood collection set in accordance with the present invention;

FIG. 7 is a perspective view of an alternate needle assembly shown in an shielding position including a tip guard;

FIG. 8 is a cross-sectional view of the needle assembly of FIG. 7;

FIG. 9 is a perspective view of a tip guard assembly for use in an alternate embodiment of the present invention;

FIG. 10 is a cross-sectional view of the tip guard assembly of FIG. 9, shown in a non-shielding position; and

FIG. 11 is a cross-sectional view of the tip guard assembly of FIG. 9, shown in a shielding position.

### DETAILED DESCRIPTION

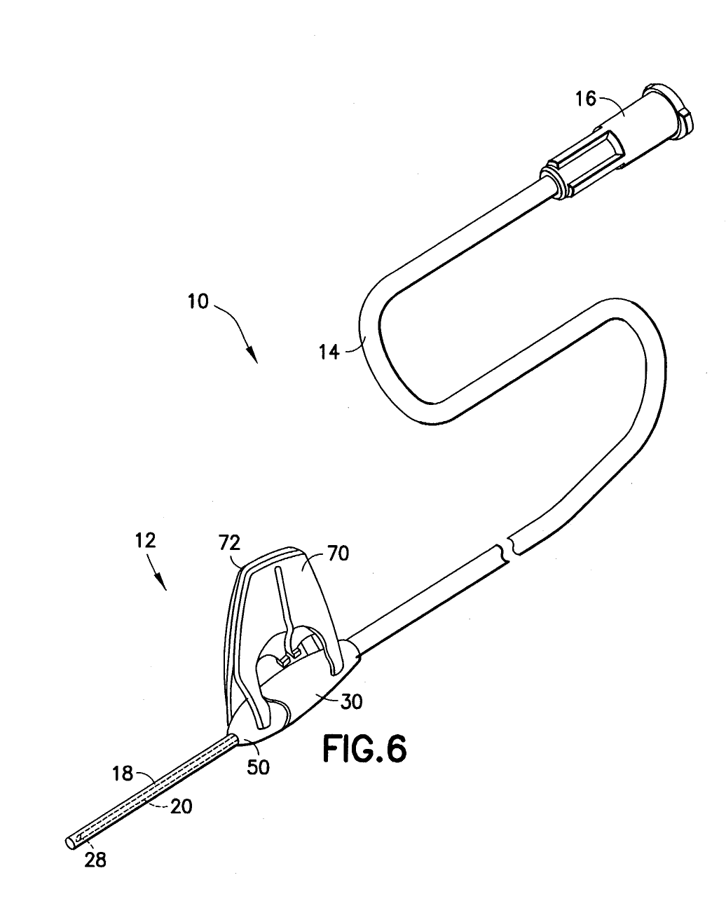

Referring to the drawings in which like reference characters refer to like parts throughout the several views thereof, FIG. 6 illustrates a blood collection set 10 in accordance with the present invention and the related features. The present invention is generally described in terms of a blood collection set, and encompasses such a blood collection set as well as a shieldable needle assembly for use in such a blood collection set.

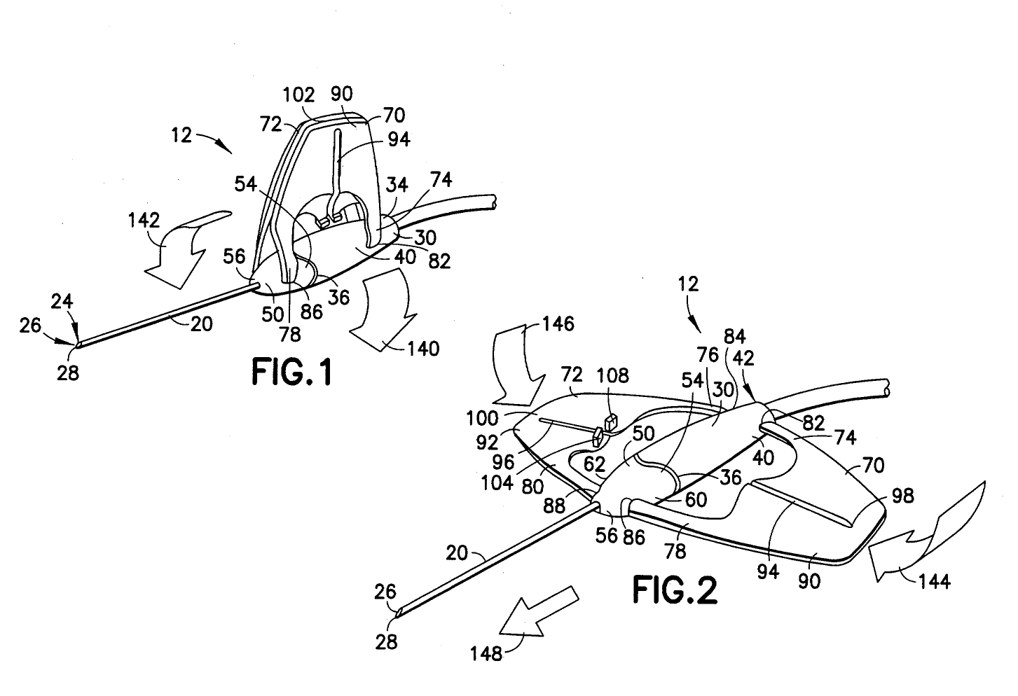

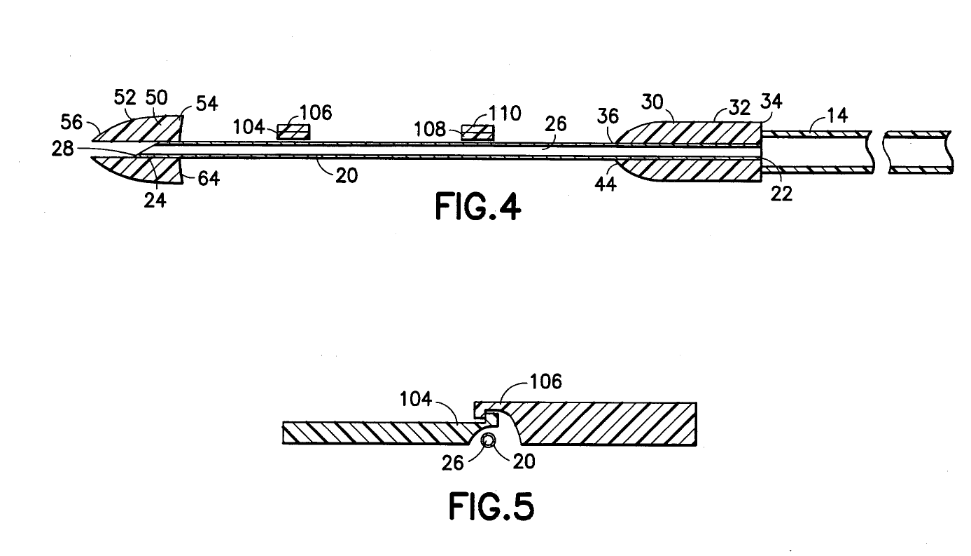

As shown in FIG. 6, blood collection set 10 includes a shieldable needle device 12, a flexible tube 14 extending from needle device 12, and a fixture 16 mounted to flexible tube 14. Shieldable needle device 12 of blood collection set 10 is shown in detail in FIGS. 1-5, and includes a needle cannula 20, a hub 30, and a shield 50. Fixture 16 is connectable to a receptacle (not shown) for use in blood collection procedures, and may therefore include appropriate structure such as a Luer type mating surface, as is known in the art.

Needle cannula 20 includes a proximal end 22 and an opposing distal end 24, with lumen 26 extending through needle cannula 20 from proximal end 22 to distal end 24. Distal end 24 of needle cannula 20 is beveled to define a sharp puncture tip 28, such as an intravenous puncture tip. Puncture tip 28 is provided for insertion into a patient's blood vessel, such as a vein, and is therefore designed to provide ease of insertion and minimal discomfort during venipuncture. A removable protective packaging cover 18 may be positioned over distal end 24 of needle cannula 20 for protection from puncture tip 28 prior to use of blood collection set 10.

Needle device 12 further includes hub 30. Hub 30 is a unitary structure, desirably molded from a thermoplastic material. Hub 30 is generally defined by an elongate tubular body in the form of housing 32 having a proximal end 34, a distal end 36, and an internal passageway 38 extending therethrough from proximal end 34 to distal end 36. Needle cannula 20 is positioned within and is supported by internal passageway 38 of hub 30, with distal end 24 of needle cannula 20 extending from distal end 36 of hub 30. Desirably, needle cannula 20 and hub 30 are separate parts which are fixedly attached and secured through an appropriate medical grade adhesive or the like.

Proximal end 34 of hub 30 is adapted for connection with a flexible tube 14 of blood collection set 10. As such, hub 30 may include any appropriate means for coupling with flexible tube 14, as is known in the art.

Needle device 12 further includes shield 50, which is defined by a housing 52. Housing 52 is a unitary structure, desirably molded from a thermoplastic material, including a proximal end 54, a distal end 56 and an internal passageway 58 extending between proximal end 54 and distal end 56.

Shield 50 co-axially surrounds a portion of needle cannula 20, and is positioned axially with respect to hub 30. In particular, shield 50 is positioned distally of hub 30, that is, beyond distal end 36 of hub 30 toward distal end 24 of needle cannula 20. Shield 50 is movable between a non-shielding or retracted position in which puncture tip 28 is exposed from distal end 56 of shield 50, and a shielding or extended position in which puncture tip 28 of needle cannula 20 is covered by shield 50, as will be discussed in more detail herein.

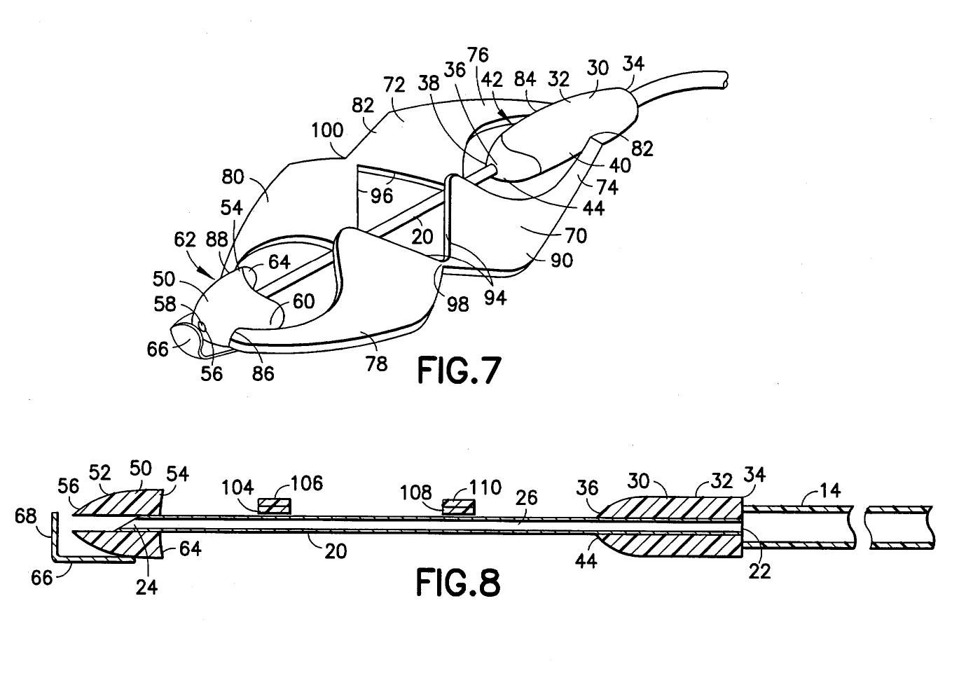

Optionally, as shown in FIGS. 7-8, a needle tip guard 66 is attached to housing 52 of shield 50 toward distal end 56 thereof. Tip guard 66 is provided to automatically cover the opening to internal passageway 58 at distal end 56 of shield 50 when shield 50 is moved to the fully extended position, covering puncture tip 28 of needle cannula 20. Tip guard 66 may be a curved leaf spring of metal or the like having an axially extending spring leg which curves generally at a right-angle to form locking plate 68. Tip guard 66 may be attached to housing 52 of shield 50 through any known means, such as through an adhesive, or through the shape and design of the tip guard, such as by a collar which extends around or into housing 52 of shield 50.

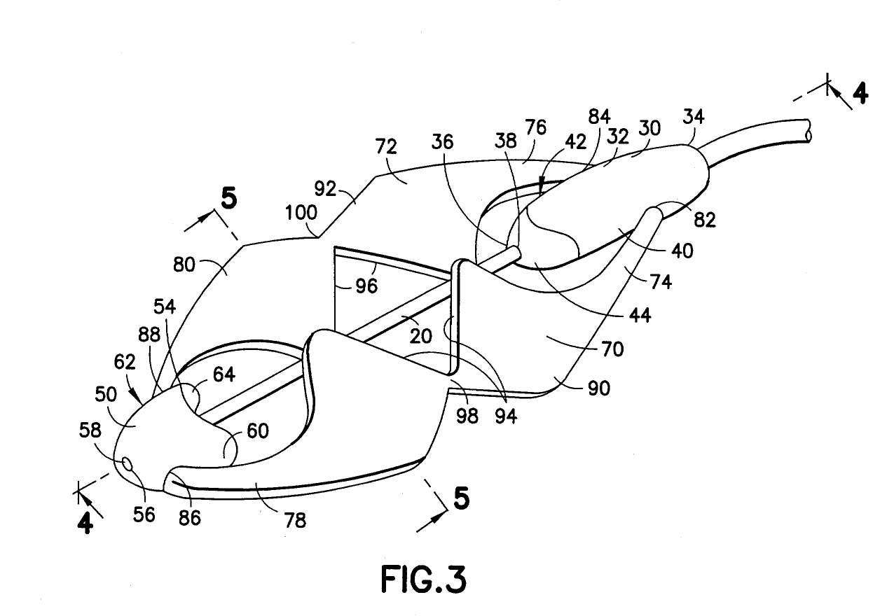

Needle device 12 also includes a pair of extendable members 70 and 72 interconnecting hub 30 and shield 50. Each of extendable members 70 and 72 includes shape and structure capable of forming at least two distinct positions. More particularly, as shown in FIG. 2, extendable members 70 and 72 are provide in a laterally spaced position forming a pair of wings extending laterally at opposing sides of needle device 12. Extendable members 70 and 72 are capable of being extended from this laterally spaced position toward the central axis of needle device 12 and toward each other to cause shield 50 to move from the non-shielding position to the shielding position, as shown in FIG. 3.

In one particular embodiment, each of extendable members 70 and 72 includes shape and structure capable of forming three distinct positions. More particularly, in a first mating position, extendable members 70 and 72 can be joined together to form a unitary structure, as shown in FIG. 1. In a second lateral position, extendable members 70 and 72 are spread apart to form a pair of wings extending laterally at opposing sides of needle device 12, as shown in FIG. 2. In a third extended position, extendable members 70 and 72 are extended to cause shield 50 to move from the non-shielding position to the shielding position, as shown in FIG. 3.

As noted, the shape and structure of extendable members 70 and 72 account for movement between these distinct positions. In particular, extendable members 70 and 72 include proximal extensions 74 and 76, respectively, and distal extensions 78 and 80, respectively. Proximal extensions 74 and 76 are separately attached to hub 30 at opposing lateral sides 40 and 42 of hub 30, respectively. Such attachment of proximal extensions 74 and 76 to hub 30 forms proximal living hinges 82 and 84, respectively, at the point of attachment of proximal extensions 74 and 76 to hub 30. In a similar manner, distal extensions 78 and 80 are separately attached to shield 50 at opposing lateral sides 60 and 62 of shield 50, respectively. Such attachment between distal extensions 78 and 80 to shield 50 forms distal living hinges 86 and 88, respectively, at the point of attachment of distal extensions 78 and 80 to shield 50.

Extendable members 70 and 72 further include extendable bridge portions 90 and 92 extending between proximal extensions 74 and 76 and distal extensions 78 and 80, respectively. Extendable bridges 90 and 92 provide the main body for the interconnection between hub 30 and shield 50, with proximal extensions 74 and 76 and distal extensions 78 and 80 providing the point of attachment to hub 30 and shield 50, respectively, for such interconnection. Each of extendable bridges 90 and 92 includes a partial cut-away portion 94 and 96 respectively. These partial cutaway portions 94 and 96 are in the form of a partial slice through the structure of each of extendable bridges 90 and 92. The partial slices through the structure thereof do not extend entirely therealong, but end at an edge portion, thereby forming two separate hinges in the form of bridge hinges 98 and 100, respectively.

As noted, such structure permits each of extendable members 70 and 72 to move between at least two, and desirably three distinct positions. In a first mating position shown in FIG. 1, extendable members 70 and 72 are joined together at a top portion of needle device 12 to form a unitary structure, such as a dorsal fin. This is easily accomplished through a bending of proximal extensions 74 and 76 at proximal living hinges 82 and 84, and distal extensions 78 and 80 at distal living hinges 86 and 88. This unitary structure provides needle device 12 with a low profile for packaging, thus reducing packaging material and expense. Also, this dorsal fin-shaped unitary structure acts as an insertion guide mechanism during venipuncture and positioning of needle device 12 within a patient, as will be described in further detail with respect to use of the needle device 12. Extendable members 70 and 72 are capable of being releasably maintained in this mating position. This may be accomplished, for example, by providing a latch mechanism at juncture 102 between extendable members 70 and 72. Desirably, extendable members 70 and 72 are releasably maintained in this first position by providing a releasable adhesive therebetween at juncture 102. Such a releasable adhesive can function to maintain extendable members 70 and 72 joined together in the first mating position, with extendable members 70 and 72 capable of being pried apart to release the adhesive connection therebetween.

Each of extendable members 70 and 72 can be moved in opposing circumferential directions about needle device 12 in a direction of arrows 140 and 142, respectively, thereby causing extendable members to be moved to a lateral position as depicted in FIG. 2. Such movement is accomplished through the hinged attachment of extendable members 70 and 72 to opposing lateral sides 40 and 42 of hub 30 and opposing lateral sides 60 and 62 of shield 50 at proximal living hinges 82 and 84 and distal living hinges 86 and 88. When in this lateral position, extendable members 70 and 72 act as a pair of stabilizers in the form of wings extending laterally from needle device 12 at opposing sides thereof, providing sampling or blood collection set 10 as a butterfly-type wing assembly. When extendable members 70 and 72 are in this lateral position forming a pair of wings, they are particularly adapted to lie flat against the surface of a patient's skin during the sampling or blood collection procedure, and may be taped to the patient's skin to maintain blood collection set 10 in place.

Alternatively, needle device 12 may be packaged in the position as shown in FIG. 2, with extendable members 70 and 72 laterally spread apart. In this case, extendable members 70 and 72 may be moved together to form a unitary structure as shown in FIG. 1 to assist in needle insertion, and then spread apart to the lateral position of FIG. 2 as described above.

As depicted in FIGS. 1 and 2, shield 50 is desirably located adjacent hub 30 when extendable members 70 and 72 are in the mated position and the lateral position. As such, distal face 44 of hub 30 directly contacts proximal face 64 of shield 50, thereby forming an axially aligned unitary structure.

Each of extendable members 70 and 72 can further be moved by applying an inward force in a direction of arrows 144 and 146, thereby causing extendable members 70 and 72 to be extended to an extended position, as shown in FIG. 3. Such extendable movement is accomplished through the cut-away portions 94 and 96 creating bridge hinges 98 and 100, respectively. By applying force in the direction of arrows 144 and 146, cut-away portions 94 and 96 are caused to be spread apart, with extendable bridges 90 and 92 extending along bridge hinges 98 and 100. Since each of extendable members 70 and 72 are connected to hub 30 and shield 50 through proximal living hinges 82 and 84 at proximal extensions 74 and 76 and through distal living hinges 86 and 88 at distal extensions 78 and 80, such extension along bridge hinges 98 and 100 causes shield 50 to be moved in a direction of arrow 148 along needle cannula 20, with hub 30 and shield 50 being moved in opposing axial directions. Thus, shield 50 is caused to be moved from the retracted or non-shielding position in which puncture tip 28 of needle cannula 20 is exposed, to the extended or shielding position, in which puncture tip 28 of needle cannula 20 is covered by shield 50. Moreover, the shape and structure of extendable members 70 and 72 prevents shield 50 from moving entirely beyond puncture tip 28 such that shield 50 is removed from needle cannula 20, thereby re-exposing puncture tip 28.

Extendable bridges 90 and 92 further include locking mechanisms for locking the pair of extendable bridges 90 and 92 in place in the extended position. For example, as shown in FIGS. 3-5, first locking mechanisms 104 and 106 may be provided at opposing portions of extendable bridges 90 and 92, respectively, and second locking mechanisms 108 and 110 may be provided at opposing portions of extendable bridges 90 and 92, respectively. Moreover, first locking mechanisms 104 and 106, as well as second locking mechanisms 108 and 110, include corresponding engaging structures so as to engage each other and lock in place, thereby preventing movement in an opposing direction. During extension of extendable bridges 90 and 92, first locking mechanisms 104 and 106 move toward each other and engage each other, while second locking mechanisms 108 and 110 also move toward each other and engage each other at a separate location. As such, extendable bridges 90 and 92 engage each other at two separate locking positions, permitting extendable members 70 and 72 to lock at two distinct places, providing improved lock strength for preventing shield 50 from being moved from the non-shielding position to the shielding position.

As shown in FIGS. 3-5, first locking mechanisms 104 and 106 and second locking mechanisms 108 and 110 may engage each other, respectively, at a position above needle cannula 20. Alternatively, extendable members 70 and 72 may be designed to include locking mechanisms which engage at any position, such as below needle cannula 20, or straddling needle cannula 20 about opposing sides thereof. In a further embodiment, it is contemplated that locking mechanisms may be provided on one or both of extendable members 70 and 72 for engagement directly with needle cannula 20.

Needle device 12 of blood collection set 10 may be constructed as a unitary member which is integrally formed including hub 30, shield 50, and extendable members 70 and 72 extending therebetween, with appropriate distinction between each of the elements as discussed above and shown in FIGS. 1-3. Alternatively, hub 30, shield 50, and extendable members 70 and 72 may be separately formed, and joined together through an appropriate adhesive. Desirably, each element is constructed of a resilient material, with extendable members 70 and 72 being constructed of a resiliently flexible material, thus providing appropriate flexibility for hinged movement of proximal living hinges 82 and 84, distal living hinges 86 and 88, and bridge hinges 98 and 100. Desirably, the components are molded of a thermoplastic polymeric material.

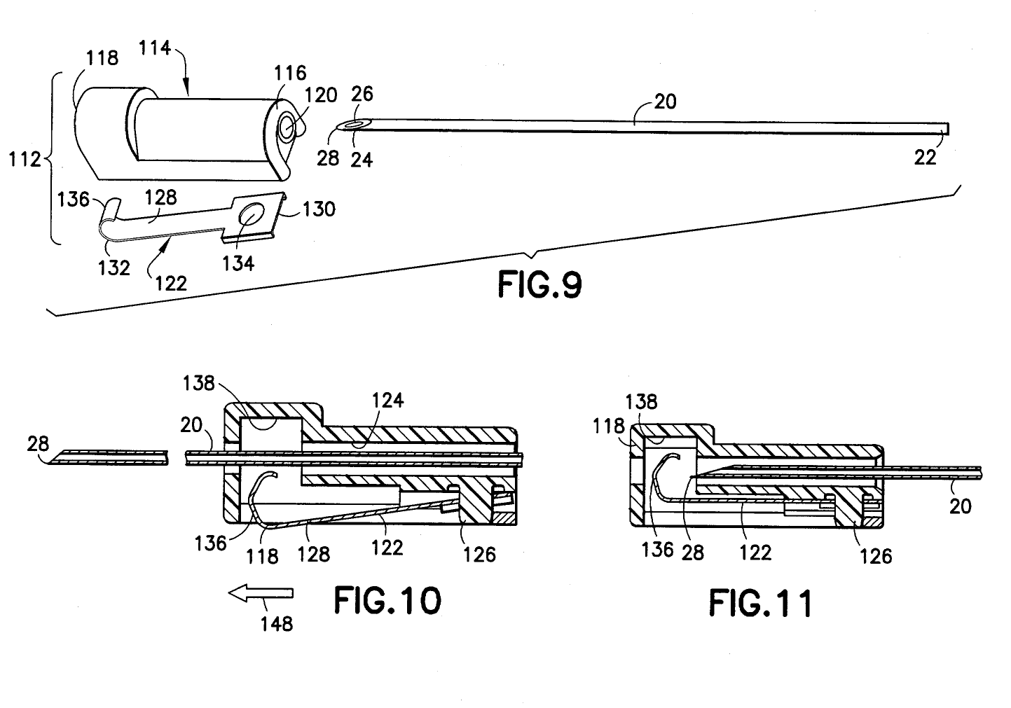

As noted above, shield 50 may optionally include tip guard 66, for covering puncture tip 28 of needle cannula 20 when shield 50 is moved to the fully extended position, as shown in FIGS. 7 and 8. In a further embodiment of the present invention depicted in FIGS. 9-11, shield 50 may be provided in the form of tip guard assembly 112, or may be provided as shield 50 including tip guard assembly 112 as a portion thereof, for engagement with puncture tip 28 when shield 50 is moved to the extended position. Tip guard assembly 112 includes a housing 114 and a protective clip 122. Housing 114 is a unitary structure, desirably molded from a thermoplastic material, including a proximal end 116, a distal end 118, and an internal passage 120 extending between the ends. Portions of internal passage 120 adjacent distal end 118 define an enlarged clip receptacle 124, as shown in FIG. 8. A clip mounting post 126 extends downwardly from housing 114 at a location near proximal end 116 of housing 114.

Protective clip 122 is unitarily stamped and formed from a resiliently deflectable metallic material. Protective clip 122 includes a planar spring leg 128 with a proximal end 130 and an opposed distal end 132. A mounting aperture 134 extends through spring leg 128 at a location near proximal end 130. Mounting aperture 134 has a diameter approximately equal to or slightly less than the diameter of mounting post 126 of housing 114. As such, mounting post 126 can be forced through mounting aperture 134 when the axis of mounting post 126 and the axis of mounting aperture 134 are substantially collinear. A lock out leg 136 extends angularly from distal end 132 of spring leg 128. Lock out leg 136 is bent back toward proximal end 130 of clip 126. The bends in lock out leg 136 enable secure protective engagement with puncture tip 28 of needle cannula 20 and further enables smooth axial sliding movement of tip guard assembly 112 along needle cannula 20.

Blood collection set 10 can be packaged substantially in the condition shown in FIG. 6. The shape and configuration of needle device 12 provides a low profile for packaging of blood collection set 10.

In use, blood collection set 10 is provided with needle device 12 assembled as described and including flexible tube 14 extending from needle device 12 and connected to fixture 16. After removing blood collection set 10 from its package, it can be assembled with other appropriate medical equipment for use. For example, an appropriate receptacle, such as a non-patient needle assembly and a needle holder, may be connected to blood collection set 10 through fixture 16, thereby providing fluid communication with lumen 26 through needle cannula 20.

To prepare for use of blood collection set 10, the user grasps blood collection set 10 at needle device 12 and removes the protective packaging cover 18 to expose puncture tip 28 of needle cannula 20. The medical practitioner can then urge puncture tip 28 at distal end 24 of needle cannula 20 into a targeted blood vessel of a patient. During such positioning, extendable members 70 and 72 of needle device 12 may be in the mating position forming a unitary structure as shown in FIG. 1 and described above. As such, the user can grasp needle device 12 at opposing sides of extendable members 70 and 72 between the user's thumb and forefinger, to facilitate positioning and placing of needle device 12 with one hand.

After venipuncture is accomplished, the user can then release the connection at juncture 102, such as by prying apart extendable members 70 and 72, if they are adhesively joined or locked together. Extendable members 70 and 72 can then be moved in opposing circumferential directions about needle device 12 in a direction of arrows 140 and 142, through the action of proximal living hinges 82 and 84 at opposing lateral sides 40 and 42 of hub 30 and distal living hinges 86 and 88 at opposing lateral sides 60 and 62 of shield 50. Such spreading apart causes extendable members 70 and 72 to form a pair of wings extending from opposing lateral sides of needle device 12. These wings formed by extendable members 70 and 72 can then be taped to the patient's skin to maintain blood collection set 10 in place during the procedure. Alternatively, extendable members 70 and 72 may automatically flex to the lateral position shown in FIG. 2 based on the resilient nature of proximal living hinges 82 and 84 at opposing lateral sides 40 and 42 of hub 30 and distal living hinges 86 and 88 at opposing lateral sides 60 and 62 of shield 50.

Upon completion of the desired procedure, such as when all desired samples have been drawn or after infusion has occurred, needle cannula 20 is withdrawn from the patient. Since extendable members 70 and 72 are connected to hub 30 and shield 50 through proximal living hinges 82 and 84 and distal living hinges 86 and 88, extendable members 70 and 72 can be brought together by moving them about proximal living hinges 82 and 84 and distal living hinges 86 and 88 in a direction opposite arrows 140 and 142 between the user's thumb and forefinger, to again form a unitary dorsal fin structure, as in FIG. 1. Needle cannula 20 can then be withdrawn from the patient.

After removal of needle cannula 20 from the patient, activation of the safety feature of needle device 12 is accomplished. In particular, the user releases the needle device 12 from the user's thumb and forefinger. The resilient nature of the structure of needle device 12 and extendable members 70 and 72 cause them to move in the direction of arrows 140 and 142, and return to the lateral position forming a pair of wings extending from opposing sides of needle assembly 12.

At this point, the user can grasp extendable members 70 and 72 at the opposing edges adjacent bridge hinges 98 and 100, between the user's thumb and forefinger. By exerting a force in the direction of arrows 144 and 146 against bridge hinges 98 and 100, i.e., by movement of bridge hinges 98 and 100 toward each other toward the central axis of needle device 10, cut-away portions 94 and 96 are expanded across bridge hinges 98 and 100, thereby causing extendable bridges 90 and 92 to extend. With the interconnection between hub 30 and shield 50 established through extendable members 70 and 72, such extension along bridge hinges 98 and 100 causes shield 50 to be moved in a direction of arrow 148 along needle cannula 20, with hub 30 and shield 50 being moved in opposing axial directions. Hence, puncture tip 28 of needle cannula 20 is safely shielded by shield 50 in the shielding position as shown in FIG. 3.

Extension of extendable bridges 90 and 92 further causes first locking mechanisms 104 and 106 and second locking mechanisms 108 and 110 to move toward each other and engage each other at two separate locking positions, thereby locking extendable members 70 and 72 in place to prevent movement in an opposing direction. Furthermore, in embodiments in which shield 50 incorporates a tip guard 66, as shown in FIGS. 7-8 described above, locking plate 68 of tip guard 66 is biased against needle cannula 20 when shield 50 is in the non-shielding position. As shield 50 is extended toward distal end 24 of needle cannula 20, tip guard 66 slides along a bottom surface of needle cannula 20 until shield 50 passes puncture tip 28. At this point, locking plate 68 is no longer in engagement with needle cannula 20, and is caused to automatically extend over the distal end 56 of shield 50, thereby preventing any re-emergence of puncture tip 28, should the locking mechanisms of extendable members 70 and 72 become disengaged. In this manner, tip guard 66 acts as a further lock for preventing exposure of puncture tip 28. Tip guard assembly 112, shown in FIGS. 9-11 as discussed above, operates in a similar manner, with movement of shield 50 to the shielding position causing protective clip 122 to extend beyond puncture tip 28 of needle cannula 20, thereby further preventing re-exposure of puncture tip 28. Blood collection set 10 may then be appropriately discarded.

While the needle assembly of the present invention has been described in terms of one embodiment for use in connection with a blood collection system, it is further contemplated that the needle assembly could be used with other medical procedures, such as in conjunction with a conventional intravenous infusion set, which are well known in the art for use with needle assemblies.

While the present invention is satisfied by embodiments in many different forms, there is shown in the drawings and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

What is claimed is:

1. A needle assembly, comprising:
a needle cannula having a proximal end and a distal end with a puncture tip;
a hub member having a proximal end and a distal end, said hub member supporting the proximal end of the needle cannula;
a shield member axially movable with respect to said hub member and said needle cannula between a non-shielding position in which said puncture tip of said needle cannula is exposed from said shield member, and a shielding position in which said shield member covers said puncture tip of said needle cannula; and
a pair of extendable members interconnecting the hub member and the shield member, said pair of extendable members movable between a lateral position in which said pair of extendable members form a pair of wings extending from opposing sides of said needle assembly, and an extended position in which said pair of extendable members are extended to cause said shield member to move from said non-shielding position to said shielding position.

2. A needle assembly as in claim 1, wherein said pair of extendable members are formed of a resilient flexible material.

3. A needle assembly as in claim 1, wherein each of said pair of extendable members includes a proximal extension and a distal extension, each of said proximal extensions of each of said pair of extendable members connected to opposing lateral sides of said hub member and each of said distal extensions of each of said pair of extendable members connected to opposing lateral sides of said shield member.

4. A needle assembly as in claim 3, wherein each of said proximal extensions of each of said pair of extendable members are connected to opposing lateral sides of said hub member and each of said distal extensions of each of said pair of extendable members are connected to opposing lateral sides of said shield member through living hinges.

5. A needle assembly as in claim 3, wherein each of said pair of extendable members includes an extendable bridge having structure capable of extending to move said shield member from said retracted position to said extended position.

6. A needle assembly as in claim 5, wherein said structure of each of said extendable bridges includes a partial cut-away portion forming a living hinge.

7. A needle assembly as in claim 6, wherein each of said extendable bridges includes first and second locking mechanisms, said first locking mechanisms engagable with each other and said second locking mechanisms engagable with each other for locking said pair of extendable members in said extended position in which said pair of extendable members are extended.

8. A needle assembly as in claim 1, wherein said pair of extendable members are movable between a mating position in which said pair of extendable members form a unitary structure, a lateral position in which said pair of extendable members are spread apart to form a pair of wings extending from opposing sides of said needle assembly, and an extended position in which said pair of extendable members are extended to cause said shield member to move from said non-shielding position to said shielding position.

9. A needle assembly as in claim 8, wherein said pair of extendable members form a dorsal fin when said pair of extendable members are in said mating position forming a unitary structure.

10. A needle assembly as in claim 8, wherein said pair of extendable members includes means for releasably maintaining said pair of extendable members in said mating position in which said pair of extendable members form a unitary structure.

11. A needle assembly as in claim 1, further comprising means for preventing said shield member from moving in an axial direction between said shielding position and said non-shielding position.

12. A needle assembly as in claim 1, wherein said shield member further comprises a tip guard including a tip guard housing formed from a plastic material, a metallic spring clip being mounted to said housing, said spring clip being biased against said needle cannula when shield member is in said non-shielding position and being resiliently moved over said distal end of said needle cannula when said shield member is in said shielding position.

13. A needle assembly as in claim 1, wherein said hub member is adapted for connection to a flexible tube of a blood collection set.

14. A needle assembly, comprising:
a needle cannula having a proximal end and a distal end with a puncture tip;
a hub member having a proximal end and a distal end, said hub member supporting the proximal end of the needle cannula;
a shield member axially movable with respect to said hub member and said needle cannula between a non-shielding position in which said puncture tip of said needle cannula is exposed from said shield member, and a shielding position in which said shield member covers said puncture tip of said needle cannula; and
a pair of extendable members interconnecting the hub member and the shield member, said pair of extendable members including structure capable of joining to form a unitary structure, spreading to form a pair of wings extending from opposing sides of said needle assembly, and extending to cause said shield member to move from said non-shielding position to said shielding position.

15. A needle assembly as in claim 14, wherein said pair of extendable members includes structure capable of joining to form a dorsal fin.

16. A needle assembly as in claim 14, wherein each of said pair of extendable members includes a proximal extension and a distal extension, each of said proximal extensions of each of said pair of extendable members connected to opposing lateral sides of said hub member and each of said distal extensions of each of said pair of extendable members connected to opposing lateral sides of said shield member through living hinges.

17. A needle assembly as in claim 14, wherein each of said pair of extendable members includes an extendable bridge having structure capable of extending to move said shield member from said non-shielding position to said shielding position.

18. A needle assembly as in claim 14, further comprising a locking assembly for preventing said shield member from moving in an axial direction between said non-shielding position and said shielding position.

19. A shieldable blood collection set comprising:
a fixture for connecting the blood collection set to a receptacle;
a flexible tube having opposed first and second ends, said first end of said flexible tube being connected to said fixture;
a hub including a housing having an outer surface, a proximal end, a distal end and a passageway extending therethrough, said proximal end of said hub mounted to said second end of said flexible tube;
a needle cannula extending from said distal end of said hub, said needle cannula including a proximal end and a distal end having a puncture tip;
a shield comprising a housing having a proximal end, a distal end and a passageway extending therethrough, said shield co-axially surrounding said needle cannula, said shield adapted for movement between a non-shielding position in which said puncture tip of said needle cannula is exposed from said distal end of said shield, and a shielding position in which said shield covers said puncture tip of said needle cannula; and
a pair of extendable members interconnecting the hub and the shield, said pair of extendable members movable between a lateral position in which said pair of extendable members form a pair of wings extending from opposing sides of said needle assembly, and an extended position in which said pair of extendable members are extended to cause said shield to move from said non-shielding position to said shielding position.

20. A blood collection set as in claim 20, wherein said pair of extendable members arc movable between a mating position in which said pair of extendable members form a unitary structure, a lateral position in which said pair of extendable members are spread apart to form a pair of wings extending from opposing sides of said needle assembly, and an extended position in which said pair of extendable members are extended to cause said shield to move from said non-shielding position to said shielding position.

21. A blood collection set as in claim 20, wherein said shield is in said non-shielding position and is adjacent said hub when said pair of extendable members are in said mating position and said lateral position, and said shield is in said shielding position when said pair of extendable members are extended to said extended position.
